(19) United States
(12) Patent Application Publication
(10) Pub. No.: US 2007/0161047 A1
(43) Pub. Date: Jul. 12, 2007

(54) Methods of labeling transiently expressed proteins in large-scale eukaryotic cell cultures

(76) Inventors: Xiaotian Zhong, Wayland, MA (US); Ronald William Kriz, Northborough, MA (US)

Correspondence Address: FITZPATRICK CELLA (WYETH), 30 ROCKEFELLER PLAZA, NEW YORK, NY, 10112-3800, US

(21) Appl. No.: 11/651,135

(22) Filed: Jan. 9, 2007

Related U.S. Application Data
(60) Provisional application No. 60/757,591, filed on Jan. 9, 2006.

(51) Int. Cl. G01N 33/53; C12P 21/06; C12N 1/18; C12N 5/06; C07K 14/47

(52) U.S. Cl. 435/7.1; 435/69.1; 435/320.1; 435/325; 435/348; 435/254.1; 435/254.2; 530/409

(57) ABSTRACT

The present invention is based on observations that transiently transfecting large-scale eukaryotic cell cultures with a polynucleotide encoding a protein of interest can be used to rapidly produce the large quantities of labeled proteins required for various biochemical techniques such as spectroscopy, microscopy, and crystallography, and applications including protein structure determination, protein tracing and/or localization, diagnostic and therapeutic applications, and affinity experiments. Thus, the present invention provides methods for rapidly producing large quantities of labeled proteins by using transient transfection of large-scale eukaryotic cell cultures, which are then grown in a chemically defined labeling medium that includes labeled amino acids. The present invention is also directed to methods of using the labeled proteins produced by the novel labeling methods for use in various techniques.

This application claims the benefit of priority from U.S. Provisional Patent Application No. 60/757,591, filed Jan. 9, 2006, the contents of which is hereby incorporated by reference herein in its entirety.

### BACKGROUND OF THE INVENTION

1. Field of the Invention

This invention relates to novel methods of labeling proteins for use in techniques such as spectroscopy, microscopy, and crystallography, and in applications including protein structure determination, protein tracing and/or localization, diagnostic and therapeutic applications, and affinity experiments. The methods disclosed herein are useful to rapidly produce sufficient quantities of labeled proteins by using transient transfection of large-scale eukaryotic cell cultures.

2. Related Background Art

The analysis of the structure of a protein often requires amino acid labeling, e.g., heavy atoms such as selenium or tellurium are incorporated into polypeptides for X-ray crystallography or spectroscopic analysis (Moroder (2005) J. Pept. Sci. 11(4):187-214). Recently, multiwavelength anomalous diffraction (MAD) phasing has been found to be a useful technique for high-throughput structure analysis, and commonly uses selenomethionine-substituted proteins (Kigawa et al. (2002) J. Struct. Funct. Genomics 1:29-35; Walsh et al. (1999) Acta Crystollographica 55D:1168-73). Additionally, nuclear magnetic resonance spectroscopy (NMR), a technique employed to aid in determining the three-dimensional structure of a protein, uses labeled proteins, e.g., those labeled with isotopes such as ¹⁵N, ¹³C, and ²H (e.g., Studts and Fox (1999) Protein Expr. Purif. 16(1):109-19). Selectively labeled proteins are used in techniques such as crystallography, spectroscopy, and microscopy, and applications including protein structure determinations, protein tracing and/or localization, diagnostic and therapeutic applications, and affinity experiments (e.g., Beilstein and Whanger (1987) J. Inorg. Biochem. 29(2):137-52; Easty et al. (1988) Electrophoresis 9(5):227-31; Stahelin (1975) Metabolism 24(4):505-15; Weiner and Thakur (2005) BioDrugs 19(3):145-63; Gruhler et al. (2005) Mol. Cell Proteomics (in press) e-published Aug. 8, 2005 as M500190-MCP200).

Although numerous molecular biology and biochemical techniques require labeled proteins, incorporating a labeled amino acid into a polypeptide chain is an extremely complex task. Producing large quantities of a labeled protein depends largely on efficient cellular uptake of the labeled amino acid, the acceptance of the labeled amino acid by the cellular translation machinery, and retention of the resultant labeled protein without significant degradation (e.g., Knowles and Ballard (1978) Br. J. Nutr. 40(2):275-87). A common cell-based method of producing proteins with selectively labeled amino acids, e.g., selenomethionine or selenocysteine, involves culturing a host cell in complete medium without inducing expression of the protein to be labeled, harvesting the host cells, and then resuspending the cells for protein expression in a labeling medium containing the labeled amino acid (e.g., Studts and Fox (2000) InNovations 10:14-16). However, this approach is not amenable to scalability and can induce growth arrest and/or loss of protein expression during harvest and wash (e.g., Studts and Fox (2000), supra). Alternatively, the host cell may be supplied with the labeled amino acid throughout growth and expression. This is a costly approach, and the presence of nonnatural amino acids for extensive periods is often cytotoxic (e.g., Kajander et al. (1991) Biol. Trace Elem. Res. 28(1):57-68).

The most common cell-based protein labeling systems use auxotrophic E. coli strains supplemented with labeled amino acids as the growth-limiting reagent (see, e.g., Sreenath et al. (2005) Protein Expr. Purif. 40(2):256-67). These E. coli-based expression systems, in which labeled amino acids substitute for unlabeled amino acids, allow selective labeling of proteins for NMR and crystal studies (e.g., Hendrickson et al. (1990) EMBO J. 9(5):1665-72). However, auxotrophic E. coli strains usually produce exogenous proteins at low levels, and many proteins cannot be expressed in a soluble form in prokaryotes. Thus, researchers often resort to using eukaryotic systems by employing host cells derived from yeast, fungi, insects or mammals (see, e.g., Bellizzi et al. (1999) Structure Fold Des. 7(11):R263-67). Although these systems overcome the solubility problems created in prokaryotic-based systems, they traditionally give low protein yields while consuming a substantial amount of time and reagents.

To increase the yield of labeled proteins in eukaryotic expression systems, researchers often create cell lines stably transfected with a polynucleotide encoding the protein of interest (see, e.g., Lustbader et al. (2005) Endocrinology 136:640-50; Hamaoka et al. (2004) Proc. Natl. Acad. Sci. USA 101:11673-78; Hendrikson et al., supra; Carfi et al. (1999) Proc. Natl. Acad. Sci. USA 96:12379-83). While transient transfection of cultures usually results in a transfection efficiency of 1-10%, i.e., 1-10% of cells in the transfected culture overexpress the protein of interest, a stably transfected cell line is clonally expanded such that all cells within the culture overexpress the desired protein. Thus, stable cell cultures generally can produce much larger quantities of a labeled protein, i.e., sufficient quantities for use in crystallography, spectroscopy, and microscopy. However, while transient transfections can be completed within a few days, the creation of stable transfectants requires several months to complete.

As an alternative to the time-consuming process of producing stable cell lines, large-scale transient transfections may be performed, e.g., transfection of 50 ml to 20 liter cultures, to produce larger quantities of a desired protein(s) in a shorter time period. However, larger cultures traditionally require larger quantities of reagents and ultra-pure plasmid DNA—a major cost factor and obstacle to protein purification (e.g., Wright et al (2003) J. Biotechnol. 102(3):211-21; Derouazi et al. (2004) Biotechnol. Bioeng. 87(4):537-45). Recent advances in transfection technology, which vary the levels of serum, culture volume, culture adhesion, and transfection vehicle, have made large-scale transient transfections feasible (e.g., Baldi et al. (2005) Biotechnol. Prog. 21:148-53; Lan Pham et al. (2003) Biotechnol. Bioeng. 84:332-42; Meissner et al. (2001) Biotechnol. Bioeng. 75:198-203; Durocher et al. (2002) Nuc. Acids Res. 30:e1-9). However, while these large-scale transient systems have been used to overexpress nonlabeled proteins, the art has not described their use to produce the large quantities of labeled proteins required for structural and other biochemical studies.

Accordingly, there is a need to develop a cost-effective and rapid method of labeling proteins in eukaryotic cells to produce the quantity of labeled protein required for use in techniques such as spectroscopy, microscopy, and crystallography, and applications including protein structure determinations, protein tracing and/or localization, diagnostic and therapeutic applications, and affinity experiments.

### SUMMARY OF THE INVENTION

The present invention provides methods for producing labeled proteins in transiently transfected large-scale cell cultures. As used herein, the phrase “labeled proteins” refers to both labeled polypeptides and labeled peptides. The present invention discloses that sufficient quantities of labeled proteins may be produced in a cost-effective manner for use in various techniques and applications that require microgram quantities of labeled proteins, e.g., techniques such as spectroscopy, microscopy, and crystallography, and applications including protein structure determinations, protein tracing and/or localization, diagnostic and therapeutic applications, and affinity experiments. As used herein, the term “spectroscopy” is used interchangeably with the terms “spectronomy” and “spectrometry.” The present invention discloses that such methods may be undertaken using transiently transfected cell cultures, and hence labeling may be completed within the course of days rather than the months required to produce stably transfected cells. As a result, the invention teaches a novel method of rapidly producing large quantities of labeled proteins by using large-scale eukaryotic cell cultures transiently transfected with a polynucleotide encoding a protein of interest.

Accordingly, one embodiment of the invention includes a method of selectively labeling a protein at a target amino acid position(s) comprising: (1) transiently transfecting a cell with a polynucleotide encoding a protein of interest; (2) contacting the cell with a labeling medium, wherein the labeling medium contains a labeled amino acid(s) to be incorporated into the protein at the target amino acid position(s); and (3) expressing the protein of interest under conditions suitable to allow incorporation of the labeled amino acid into the target amino acid position(s). In another embodiment, the method further comprises the step of harvesting the labeled protein.

Labeled amino acids used in the disclosed methods may be derived from any naturally occurring amino acid, e.g., L-tyrosine, or any nonnatural amino acid, e.g., 3-iodo-L-tyrosine. In one embodiment of the invention, the labeled amino acid contains a label selected from: (1) a heavy atom label (e.g., selenium, tellurium, gold, iodine, lead, uranium, mercury, platinum, etc.); (2) a fluorescent, chemiluminescent, or photolabile label (e.g., isocyanates, isothiocyanates and benzoyl-linked amino acids); (3) an isotope label (e.g., ¹⁵N, ¹³C, ²H, ¹⁴C); and (4) a spin label (e.g., nitroxide derivatives contained within a pyrrole ring). In one embodiment of the invention, the labeled amino acid contains a heavy atom; in a further embodiment, the heavy atom label is selenium.

The present invention teaches that the disclosed protein labeling methods may be carried out in various eukaryotic cell-based systems. Thus, the invention contemplates the use of various eukaryotic cell types, including, but not limited to, host cells derived from mammals, insects, fungi, and yeast. In one embodiment of the invention, the host cell is derived from a mammal, e.g., the HEK293 or CHO cell lines. The host cells used in the disclosed method may be seeded for transfection at varying densities, e.g., about 1×10⁵ to about 3×10⁶ cells/ml culture medium, e.g., about 1.5×10⁵ to about 2×10⁶ cells/ml culture medium, depending on culture conditions and cell type. For example, in one embodiment of the invention, HEK293 host cells are plated at a density of about 0.5-1.0×10⁶ cells/ml for transfection. The culture volume during the transfection step may also be varied depending on the amount of labeled protein required. As such, the transfection volume of the large-scale cell culture varies from about 50 ml to 20 liters. In one embodiment of the invention, the culture volume of the large-scale cell culture in the transfecting step is between about 50 ml and about 20 liters or more. In other embodiments of the invention, the culture volume of the large-scale cell culture is between about 100 ml and about 20 liters or more; or between about 250 ml and about 20 liters or more; or between about 500 ml and about 20 liters or more; or between about 1 liter and about 20 liters or more; or between about 5 liters and about 20 liters or more; or between about 10 liters and about 20 liters or more; or between about 15 liters and about 20 liters or more. In one embodiment of the invention, the culture volume of the large-scale cell culture in the transfecting step is greater than about 50 ml. In another embodiment, the culture volume of the large-scale cell culture in the transfecting step is about 1 liter. In another embodiment, the culture volume of the large-scale cell culture in the transfecting step is greater than about 1 liter. In a further embodiment, the culture volume of the large-scale cell culture in the transfecting step is greater than about 20 liters; for example, culture volumes of about 40, 60, 80 or 100 liters or more are also contemplated in the present invention.

Those skilled in the art will recognize that the mode of transfecting a polynucleotide into a host cell will depend on the host cell chosen and various preferred culture conditions. Thus, transfection may be achieved by numerous vehicles and reagents that are well known in the art, including, but not limited to, liposomes, polyethylenimine, electroporation, gene guns, calcium phosphate precipitation, episomes, nanoparticles, DEAE-dextran, etc. In one embodiment of the invention, the transfection-mediating reagent is polyethylenimine. Further, the length of time required to efficiently transfect a particular host cell will also vary depending on cell type, culture media, and the transfection-mediating reagent or vehicle chosen. In some embodiments of the invention, the transfecting step is carried out for a period of time from about 24 hours to greater than 100 hours (e.g., about 24-144 hours). In other embodiments, the transfecting step is carried out for a period of time less than about 24 hours (e.g., as little as several hours). In one embodiment of the invention, the transfecting step is carried out for about 24-72 hours. In another embodiment of the invention, the labeling medium is supplemented with serum at a concentration of about 0.0001% to about 10%. In another embodiment of the invention, the labeling medium is supplemented with serum at a concentration of about 5 to about 10%. Choosing optimal parameters (such as those related to transfection reagent, culture medium, cell density and length of transfection time) for transfecting eukaryotic cells is commonly undertaken in cell and molecular biology, and is well within the knowledge of one skilled in the art.

Following transfection of the polynucleotide encoding the protein of interest into the host cell, the host cell is contacted with a labeling medium. In one embodiment of the invention, the contacting step is carried out in a labeling medium that substantially lacks the nonlabeled form of the labeled amino acid, such that the source of the amino acid to be substituted into the protein at target amino acid positions is the labeled amino acid supplied in the labeling medium. As used herein, the phrase “target amino acid positions” refers to either all or some of the sites occupied by a particular amino acid residue in a protein of interest. For example, if the labeled amino acid employed in the disclosed method is selenomethionine, and one wishes to substitute selenomethionine for all methionine residues, then the phrase “target amino acid positions” is used to refer to all methionine sites in the protein; alternatively, if one wishes to substitute selenomethionine for only some methionine residues in the protein, then the phrase “target amino acid positions” is used to refer to some methionine sites in the protein.

After the host cell containing the polynucleotide encoding the protein to be labeled is contacted with the labeling medium, the protein is expressed for a period of time to allow incorporation of the labeled amino acid into the target amino acid positions. In some embodiments of the invention, the host cells are allowed to express the protein for a period of time from about 24 hours to greater than 100 hours (e.g., about 24-144 hours). In one embodiment of the invention, the expressing step is carried out for about 48-144 hours. This expressing step typically takes place at a physiological temperature that is chosen based on culture conditions and the host cells used to express and label the desired protein. In one embodiment of the invention, the expressing step occurs at about 30-38° C.; in a further embodiment, the expressing step occurs at about 31° C.

Using the method disclosed herein, within a few days to a week it is possible to obtain about 30 μg to about 10 μg of labeled protein per culture liter, with incorporation of the labeled amino acid at about 50% to about 95% of all corresponding amino acid positions within the protein. This amount of labeled protein is suitable for numerous biochemical techniques, e.g., spectroscopy, microscopy, and crystallography, and applications including protein structure determinations, protein tracing and/or localization, diagnostic and therapeutic applications, and affinity experiments. In one embodiment of the invention, the labeled protein is used for spectroscopy, microscopy, or crystallography studies, e.g., X-ray diffraction, X-ray absorption, multiwavelength anomalous dispersion (MAD), single-wavelength anomalous dispersion (SAD), multiple isomorphous replacement (MIR), electron paramagnetic resonance (EPR), nuclear magnetic resonance (NMR), mass spectrometry (MS), circular dichroism (CD), electron spin resonance (ESR), surface plasmon resonance (SPR), electron nuclear double resonance (ENDOR), electron-electron double-resonance (ELDOR), electron spin-echo-envelope-modulation (ESEEM), Raman spectroscopy (RS), electron microscopy (EM), fluorescence correlation spectroscopy (FCS), confocal microscopy (CF), immunofluorescence microscopy (IF), fluorescence resonance energy transfer microscopy (FRET), hyperfine sublevel correlation spectroscopy (HYSCORE), fluorescence lifetime image microscopy (FLIM), fluorescent speckle microscopy (FSM), total internal reflection fluorescence microscopy (TIRF), positron emission tomography (PET), Sidec electron tomography (SET), and atomic force microscopy (AFM).

In another embodiment of the invention, a labeled protein produced by the labeling methods described herein is used for protein tracing and/or protein localization. In yet a further embodiment of the invention, a labeled protein produced by the disclosed methods is used for affinity experiments, therapeutic or diagnostic applications, or protein structure determinations.

Other features and advantages of the invention will be apparent from the following detailed description and claims.

BRIEF DESCRIPTION OF THE DRAWINGS

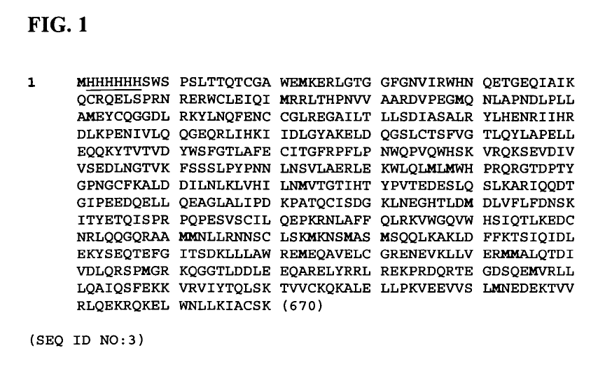

FIG. 1: Amino acid sequence of histidine-tagged human cytosolic kinase IKK2(ΔK664). The six histidine (H) tags are underlined, and the twenty methionine (M) positions are shown in bold.

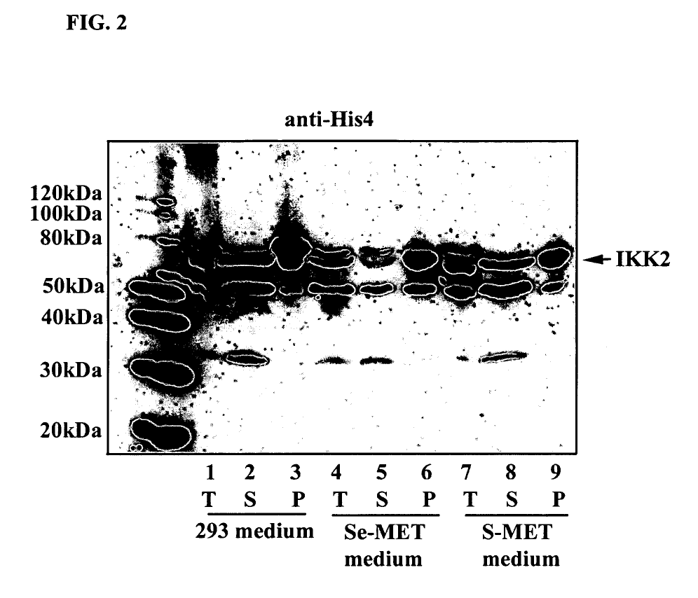

FIG. 2: Soluble IKK2(ΔK664) is produced in selenomethionine (Se-MET)-labeling media. The IKK2(ΔK664) protein was transiently expressed into HEK293-EBNA cells. Cells were precipitated and resuspended in either 293 medium, labeling medium with Se-MET (selenomethionine), or labeling medium with S-MET (sulfur-containing methionine). The samples were then separated by SDS-PAGE and immunoblotted with mouse monoclonal anti-His4 antibody. Total lysates (T); supernatants (S); pellets (P).

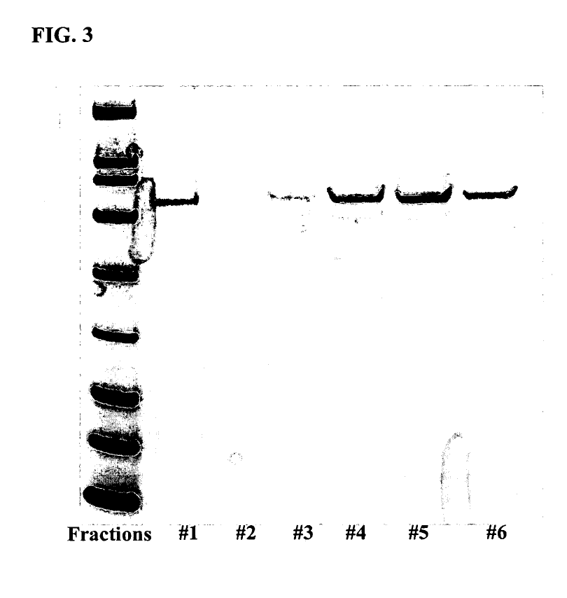

FIG. 3: Purification of Se-MET-labeled IKK2(ΔK664) from HEK293 cultures. Se-MET-labeled IKK2(ΔK664) was created as described herein and purified using column chromatography. Different elution fractions from a MonoQ column were separated by SDS-PAGE and stained with Coomassie Brilliant Blue.

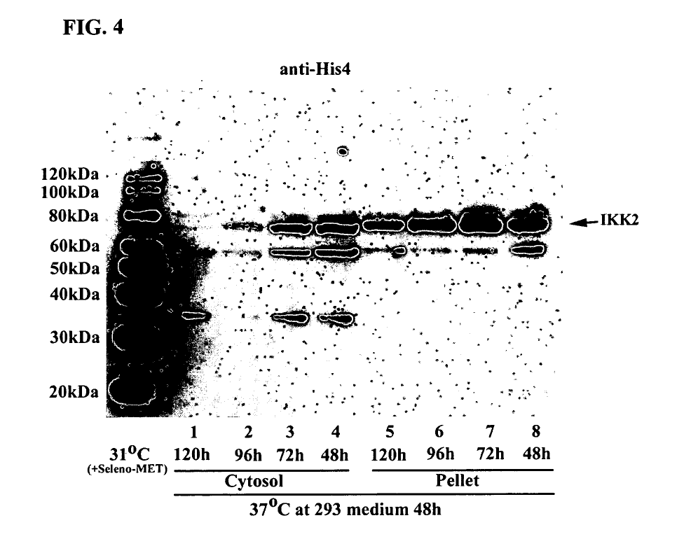

FIG. 4: Optimizing protein yields of soluble Se-MET-labeled IKK2(ΔK664) in HEK293 cells. IKK2(ΔK664) was transiently expressed in HEK293-EBNA cells and labeled as described herein. To improve protein yields, the transfected cells were resuspended in Se-MET-labeling medium supplemented with 10% FBS, and the cultures were grown at 31° C. for 48-120 hours. Protein samples were analyzed by SDS-PAGE and immunoblotted with mouse monoclonal anti-His4 antibody.

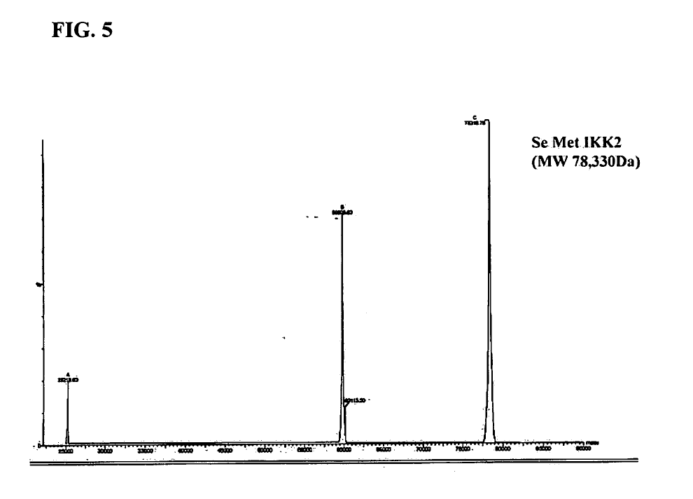

FIG. 5: Mass spectrum analysis of the Se-MET-labeled IKK2(ΔK664). The Se-MET-labeled IKK2(ΔK664) protein, created as described herein, was analyzed by HPLC ESI-Mass Spectrum. The molecular weight (MW) of Se-MET IKK2(ΔK664) is 78,330 Da, whereas the MW of S-MET IKK2(ΔK664) is 77,410 Da.

### DETAILED DESCRIPTION OF THE INVENTION

As described previously, there is a need to develop a rapid (and cost-effective) method of labeling large quantities of proteins in eukaryotic cells for use in techniques such as spectroscopy, microscopy, and crystallography, and applications including protein structure determinations, protein tracing and/or localization, diagnostic and therapeutic applications, and affinity experiments.

The present invention provides methods for producing labeled proteins in transiently transfected large-scale eukaryotic cell cultures. The present invention discloses methods for producing sufficient quantities of labeled proteins, which may be produced in a rapid and cost-effective manner, for use in various techniques that require microgram quantities of labeled proteins, e.g., techniques such as spectroscopy, microscopy, and crystallography, and applications including protein structure determinations, protein tracing and/or localization, diagnostic and therapeutic applications, and affinity experiments. These protein-labeling methods may be undertaken using transiently transfected eukaryotic cell cultures, and hence labeling may be completed within the course of days rather than the months required to produce stably transfected cells. As a result, the invention teaches a novel method of producing large quantities of labeled proteins by using large-scale eukaryotic cell cultures transiently transfected with a polynucleotide encoding a protein of interest.

Accordingly, one embodiment of the invention includes a method of selectively labeling a protein at a target amino acid position(s) comprising: transiently transfecting a cell with a polynucleotide encoding a protein of interest; contacting the cell with a labeling medium, wherein the labeling medium contains a labeled amino acid(s) to be incorporated into the protein at a target amino acid position(s); expressing the protein of interest under conditions suitable to allow incorporation of the labeled amino acid into the target amino acid position(s); and harvesting the labeled protein.

Using the disclosed labeling methods, it is possible to label a protein at all, substantially all, or some target amino acid positions, e.g., all, substantially all, or some methionine positions, in a protein of interest. It will be understood by one skilled in the art that any labeled amino acid may be used to substitute for the corresponding unlabeled amino acid in a protein of interest using the methods described herein.

Source of Amino Acids

The labeled amino acids used in the disclosed labeling method need not be derived solely from the naturally occurring amino acids. Thus, a labeled amino acid may be derived from a natural amino acid, such as L-tyrosine, an isomer of a natural amino acid, such as D-tyrosine, or a nonnatural amino acid, such as 3-iodo-L-tyrosine (see, e.g., Kobayashi et al. (2005) Proc. Natl. Acad. Sci. USA 102:1366-71; Takayama et al. (2005) Biosci. Biotechnol. Biochem. 69(5):1040-41). Labeled amino acids may be derived from amino acid analogs including, e.g., thiazolylanine, triazolalanine, dihydroxyphenylalanine, 1-pipecolate, (+/−)-nipecotic acid, S-Ethyl 2-azidohexanethioate, N-(methylamino)isobutyric acid, azetidine-2-carboxylic acid, iodo-L-alpha-methyltyrosine, alanosine, thioproline, iodo-alpha-methyl-L-tyrosine, amino-isobutyric acid, 1,3-thiazolidine-4-carboxylic acid [thiaproline, Pro(S)], p-(4-hydroxybenzoyl)phenylalanine, pipecolic acid, azetidine-2-carboxylic acid, canavanine, indospicine, triazolalanine, 2-, 3- and 4-fluorophenylalanine, 3,4-dehydroproline, beta-2-thienylalanine, dihydroxyphenylalanine, histidinol, indospicine, selenomethionine, nitrobenzoxadiazoyl-diaminopropionic acid, selenocysteine, and 5- and 6-fluorotryptophan (e.g., Turcatti et al. (1997) Receptors Channels 5:201-07; Knowles and Ballard (1978) Br. J. Nutr. 40(2):275-87). Additional amino acid analogs include, e.g., N-methylated and N-ethylated amino acid analogs disclosed in Janecka et al. ((2005) Peptides 27(1):131-35, Aug. 8, 2005 [Epub ahead of print]), and analogs disclosed in U.S. Pat. No. 5,120,859. These references, along with all other documents and references cited herein, are hereby incorporated by reference in their entireties. It will be understood by one skilled in the art that any amino acid, natural or nonnatural, is useful in the disclosed labeling methods if that amino acid is capable of being incorporated into the protein of interest during its expression in the chosen host cell.

Types of Amino Acid Labels

Numerous types of labeled amino acids are useful in the labeling methods disclosed herein. Labels present on amino acids used in the disclosed methods include, but are not limited to, (1) heavy atom labels, (2) fluorescent, chemiluminescent, or photolabile labels, (3) isotope labels, and (4) spin labels. The type of labeled amino acid chosen to label a protein of interest will depend on the proposed use for the labeled protein. Thus, one of skill in the art will recognize that the disclosed labeling methods may employ any type of detectably labeled amino acid that is capable of being incorporated into the protein of interest, and is not limited to those labels or labeled amino acids specifically noted herein.

In one embodiment of the invention, the labeled amino acid contains a heavy atom label. As used herein, the phrase “heavy atom” refers to elements with an atomic number of at least 20, and includes metals, gases, transition metals and metalloids (e.g., selenium, tellurium, gold, iodine, lead, uranium, mercury, platinum, xenon, etc. and related compounds) (e.g., Bolles et al. (1997) SAAS Bull. Biochem. Biotechnol. 10:13-17; Qoronofleh et al. (1995) J. Biotechnol. 39(2):119-28; Vitali et al. (1991) Appl. Cryst. 24: 931-35; Islam et al. (1998) Acta Cryst. D54:1199-1206; Hendrickson et al., supra). Heavy atom labels also include transition organometallic labels (e.g., imidoesters, pyrylium ions, Fischer metallo-carbenes) such as those disclosed in Salmain and Jaouen ((2003) C.R. Chimie 6:249-58). Numerous heavy atoms and reagents containing heavy atoms for use in spectroscopy and crystallography techniques are disclosed in Islam et al. (supra), which is incorporated herein by reference in its entirety.

Amino acids used in the present invention also include spin-labeled amino acids such as, e.g., Fmoc-POAC and TOAC (disclosed in Tominaga et al. (2001) Chem. Pharm. Bull. (Tokyo) 49(8):1027-29), 2,2,6,6,-tetramethyl-piperidine-N-oxyl-4-amino4-carboxylic acid (disclosed in Karim et al. (2004) Proc. Natl. Acad. Sci. USA 101(40):14437-42), and those disclosed in Zhleva et al. (1995) Pharmazie. 50(1):25-26. Additional spin labels include N-4-(2,2,6,6-tetramethylpiperidinyl-1-oxyl-4-yl) maleimide (MAL-6) and succinimidyl-2,2,5,5-tetramethyl-3-pyrroline-1-oxyl-3-carboxylate (Singh et al. (1995) Arch. Biochem. Biophys. 320(1):155-61), as well as those disclosed in Fajer, “Electron Spin Resonance Spectroscopy Labeling in Peptide and Protein Analysis” pp. 5725-61 in Encyclopedia of Analytical Chemistry, ed. Meyers, Wiley & Sons (2000). Traditional spin labels are comprised of small stable organic radicals, e.g., nitroxide derivatives containing an unpaired electron in the pII orbital of the N—O bond, with limited flexibility due to the enclosure of the radical within an environment of limited flexibility, e.g., within a piperidine or pyrrole ring. Spin-labeled proteins produced by the methods disclosed herein are useful in the investigation of molecular orientation, molecular dynamics, ligand binding, intra- and intermolecular distance measurements, and the determination of various levels of protein structure, as described and discussed in Fajer, supra, which is incorporated herein by reference.

Amino acid labels may also include fluorescent, chemiluminescent, or photolabile labels. These labels include labels of amino groups (such as arylsulfonyl halydes, isocyanates and isothiocyanates, nitrobenzoxadialoles, N-succinimides (such as Cy5 and Cy3) and anhydrides), labels of thiol groups (such as haloacetamides, maleimides, aziridines, disulfides bimanes), sufoindocyanine dyes, fluorescamine, and labels such as those set forth in Maruyama et al. (1998) Plant Cell Phys. 39(10):1045-53; Kenworth (2001) Methods 24:289-96; Mujumdar et al. (1993) Bioconjugate Chem. 4:105-11; and Tsien and Waggoner, Handbook of Confocal Microscopy, (2nd Edition) ed. Pawley, Plenum Press, NY (1994), all of which are incorporated herein by reference. In addition, amino acids useful in the present invention include sulfur-containing excitatory amino acids, i.e., homocysteine sulfinic acid (HCSA), homocysteic acid (HCA), cysteine sulfinic acid (CSA), and cysteic acid (CA), which may be derivatized with, e.g., 5-carboxy-fluorescein succinimidyl ester (Becker et al. (2002) Electrophoresis 23(15):2457-64). Other fluorescent, chemiluminescent, or photolabile labeled amino acids include: 6-fluoro-L-m-tyrosine, N alpha-(4-azidotetrafluoro-benzoyl)tryptophan, N alpha-(1-ethyl-2-diazomalonyl)-5-bromotryptophan, benzoylphenylalanine, p-(4-hydroxybenzoyl) phenylalanine, luciferin-labeled amino acids, and 2,3-dihydrophthalazinediones (e.g., luminal-labeled amino acids (Li et al. (1995) Biochem. J. 308 (Pt. 1):251-60; Nahmias et al. (1995) Movement Disorders 10(3): 298-304; Wilson et al. (1997) Biochemistry 36(15):4542-51)).

Labels may also include members of a signal producing system that act concertedly with one or more additional members of the same system to provide a detectable signal. For example, labels may include, e.g., biotin, fluorescein, digoxigenin, polyvalent cations, chelator groups and the like, where such members specifically bind to an additional member(s) of the system (such as an enzymatic moiety capable of converting substrate to a chromogenic product, e.g., an alkaline phosphatase conjugated antibody), resulting in a detectable signal either directly or indirectly.

Amino acid labels used in the disclosed methods also include radiolabels and isotope labels. Although the isotopes ¹⁵N, ¹³C, and ²H are most commonly used in spectroscopy studies, numerous other isotopes (e.g., ¹⁸F, ¹²³I, ⁷⁵Se, ³⁵S, ¹⁴C, ³H) are useful labels in a variety of techniques and applications, such as protein structure determinations, protein tracing and/or localization, diagnostic and therapeutic applications, and affinity experiments as described herein and disclosed in the literature (see, e.g., Hountondji et al. (2000) J. Protein Chem. 19(7):563-68; Gruhler et al., supra; Wiener et al. (2005) Biodrugs 19(3):145-63; Easty et al., supra; Patel et al. (1988) Electrophoresis 9(9):547-54; Kirkpatrick et al. (1985) Pediatr. Res. 19(112):1341-45; Stahelin, supra; Camier et al. (1986) FEBS Lett. 196(1):14-18; Dadoune et al. (1985) Arch. Androl. 14(2-3):199-207; Mitch and Clark (1983) Kidney Int. Suppl. 16:Supp. 2-8; Holstege and Kuypers (1982) Prog. Brain Res. 57:145-75).

One of skill in the art will understand that any one or several types of labeled amino acids may be used to label a protein of interest.

Expression Constructs and Generation of Recombinant Host Cells

The present invention uses constructs, in the form of plasmids, vectors, and transcription or expression cassettes, comprised of at least one polynucleotide encoding a protein of interest, i.e., a protein to be labeled. Vectors are capable of directing the expression of genes to which they are operably linked. Such vectors are referred to herein as “recombinant expression vectors” or “expression vectors.” In general, expression vectors of utility in recombinant DNA techniques are often in the form of plasmids. In the present specification, “plasmid” and “vector” may be used interchangeably as the plasmid is the most common vector form. However, the invention is intended to include other forms of expression vectors that serve equivalent functions, including, but not limited to, viral vectors (e.g., replication defective retroviruses, modified alphaviruses, adenoviruses and adeno-associated viruses).

Constructs that are suitable for expression of proteins in eukaryotic cells are well known in the art. For example, polynucleotides may be operably linked to an expression control sequence, such as those present in the pMT2 or pED expression vectors disclosed in, e.g., Kaufman et al. (1991) Nucleic Acids Res. 19:4485-90. Other suitable expression control sequences are found in vectors known in the art and include, but are not limited to: HaloTag™ pHT2, pACT, pBIND, pCAT®3, pCI, phRG, phRL (Promega, Madison, Wis.); pcDNA3.1, pcDNA3.1-E, pcDNA4/HisMAX, pcDNA4/HisMAX-E, pcDNA3.1/Hygro, pcDNA3.1/Zeo, pZeoSV2, pRc/CMV2, pBudCE4 pRc/RSV (Invitrogen, Carlsbad, Calif.); pCMV-3Tag Vectors, pCMV-Script® Vector, pCMV-Tag Vectors, pSG5 Vectors (Stratagene, La Jolla, Calif.); pDNR-Dual, pDNR-CMV (Clonetech, Palo Alto, Calif.); and pSMEDA (Wyeth, Madison, N.J.). General methods of expressing recombinant proteins are also known and are exemplified in, e.g., Kaufman (1990) Meth. Enzymol. 185:537-66.

As defined herein “operably linked” means enzymatically or chemically ligated to form a covalent bond between the polynucleotide to be expressed and the expression control sequence in a manner that the encoded protein is expressed by the transfected host cell.

The recombinant expression constructs of the invention may carry additional sequences, such as regulatory sequences (i.e., sequences that regulate either vector replication, e.g., origins of replication, transcription of the nucleic acid sequence encoding the polypeptide (or peptide) of interest, or expression of the encoded polypeptide), tag sequences such as histidine, and selectable marker genes. The term “regulatory sequence” is intended to include promoters, enhancers and any other expression control elements (e.g., polyadenylation signals, transcription splice sites) that control transcription, replication or translation. Such regulatory sequences are described, for example, in Goeddel, Gene Expression Technology: Methods in Enzymology, Academic Press, San Diego, Calif. (1990). Those skilled in the art will recognize that the design of the expression vector, including the selection of regulatory sequences, will depend on various factors, including choice of the host cell and the level of protein expression desired. Preferred regulatory sequences for expression of proteins in mammalian host cells include viral elements that direct high levels of protein expression, such as promoters and/or enhancers derived from the FF-1a promoter and BGH poly A, cytomegalovirus (CMV) (e.g., the CMV promoter/enhancer), Simian virus 40 (SV40) (e.g., the SV40 promoter/enhancer), adenovirus (e.g., the adenovirus major late promoter (AdMLP)), and polyoma. Viral regulatory elements, and sequences thereof, are described in, e.g., U.S. Pat. Nos. 5,168,062; 4,510,245; and 4,968,615, all of which are incorporated herein by reference.

Suitable vectors, containing appropriate regulatory sequences, including promoter sequences, terminator sequences, polyadenylation sequences, enhancer sequences, marker genes and other sequences as appropriate, may be either chosen or constructed. Inducible expression of proteins, achieved by using vectors with inducible promoter sequences, such as tetracycline-inducible vectors, e.g., pTet-On™ and pTet-Off™ (Clontech, Palo Alto, Calif.), may also be used in the disclosed method. For further details regarding expression vectors, see, for example, Molecular Cloning: a Laboratory Manual (2nd ed.) eds. Sambrook et al., Cold Spring Harbor Laboratory Press, Cold Spring Harbor, N.Y. (1989). Many known techniques and protocols for manipulation of nucleic acids, for example, in preparation of nucleic acid constructs, mutagenesis, sequencing, introduction of DNA into cells, gene expression, and analysis of proteins, are also described in detail in Current Protocols in Molecular Biology (2nd ed.) eds. Ausubel et al., Wiley & Sons, Alameda, Calif. (1992).

A polynucleotide inserted into an expression construct for producing labeled proteins may encode any protein that is capable of being expressed in the host cell used to perform the labeling. Thus, the polynucleotide may encode full-length gene products, portions of full-length genes, peptides, or fusion proteins. Such polynucleotides may consist of genomic DNA or cDNA, and may be derived from either a prokaryotic or a eukaryotic organism. Polynucleotides may be isolated from cells or organisms by methods well known in the art, e.g., PCR or RT-PCR, or may be produced by known conventional chemical synthesis methods. Such chemically synthetic polynucleotides may possess biological properties in common with the natural polynucleotides, and thus may be employed as substitutes for the natural polynucleotides. Alternatively, synthesized polynucleotides may encode proteins that differ from the natural proteins, and thus may be employed to analyze the effect of structural changes of the labeled protein.

The present invention uses recombinant host cells, i.e., cells transfected with an expression construct containing a polynucleotide that encodes a protein of interest. A number of cell lines are suitable host cells for recombinant expression of labeled proteins. Mammalian host cell lines include, for example, COS, CHO, 293T, A431, 3T3, CV-1, C3H10T1/2, Colo205, HEK293, HeLa, L cells, BHK21, HL-60, U937, HaK, Jurkat cells, Rat2, BaF3, 32D, FDCP-1, PC12, M1x or C2C12 cells, as well as transformed primate cell lines, normal diploid cells, and cell strains derived from in vitro culture of primary tissue and primary explants. Any eukaryotic cell that is capable of expressing the protein to be labeled may be used in the disclosed labeling methods. Numerous cell lines are available from commercial sources such as the American Type Culture Collection (ATCC).

Alternatively, it may be possible to recombinantly produce labeled proteins in lower eukaryotes such as yeast or fungi. Potentially suitable yeast strains include Saccharomyces cerevisiae, Schizosaccharomyces pombe, Kluyveromyces strains, and Candida strains. If labeled proteins are made in yeast or fungus, it may be necessary to modify them by, for example, phosphorylation or glycosylation of appropriate sites, in order to obtain functional labeled proteins. Such covalent attachments may be accomplished using well-known chemical or enzymatic methods.

Labeled proteins may also be recombinantly produced by operably linking the polynucleotide encoding the protein to be labeled to suitable control sequences in one or more insect expression vectors, such as baculovirus vectors, and employing an insect cell expression system. Materials and methods for baculovirus/Sf9 expression systems are commercially available in kit form (e.g., the MAXBAC® kit, Invitrogen, Carlsbad, Calif.).

Transfection of host cells with the expression construct may be achieved by numerous methods that are well known in the art. The methods disclosed herein use transient transfection rather than stable transfection in order to rapidly produce labeled proteins. Several different well-established methods exist for the delivery of molecules, particularly nucleic acids, into eukaryotic cells. Depending on the cell type and the specific experimental requirements, such as transfection of difficult cell lines or primary cells, the type of molecule transfected (genomic DNA, DNA, oligonucleotides), or the expression construct chosen, each transfer method possesses advantages and disadvantages. Common transfection methods include, e.g., calcium phosphate precipitation, liposome mediated transfection, DEAE-dextran-mediated transfection, gene guns, electroporation, nanoparticle delivery, polyamines, episomes, and polyethylenimines. In addition, numerous transfection kits and reagents are commercially available from companies such as Invitrogen (VOYAGER™, LIPOFECTIN®), EMD Biosciences, San Diego, Calif. (GENEJUICE™), Qiagen, Germantown, Md. (SUPERFEC™), Orbigen, San Diego, Calif. (SAPPHIRE™), and many others known to those of skill in the art. Transfection protocols may also be found in Basic Methods in Molecular Biology (2^{nd} ed.) eds. Davis et al., Appleton and Lange, Conn. (1994).

The present invention uses large-scale transient transfections of eukaryotic cells to produce sufficient quantities of labeled proteins required for various biochemical techniques. Methods for large-scale transient transfections are disclosed in Large-scale Mammalian Cell Culture Technology (Biotechnology and Bioprocessing Series) ed. Lubiniecki, Marcel Dekker, NY (1990); Kunaparaju et al. (2005) Biotechnol. Bioeng. 91:670-77; Maiorella et al. (1988) Bio/Technology 6:1406-10; Baldi et al., supra; Lan Pham et al., supra; Meissner et al., supra; Durocher et al., supra). In general, large-scale transient gene expression in mammalian cell cultures may employ any one of several common types of transfection modes, including, but not limited to, polyethylenimine (PEI), electric field pulse, CALFECTION™ or calcium phosphate, to achieve high transfection efficiency at scales or volumes, e.g., greater that 10 liters (Derouazi et al., supra; Rols et al. (1992) Eur. J. Biochem. 206(1):115-21; Wurm and Bernard (1999) Curr. Opin. Biotechnol. 10(2):156-59; Schlaeger and Christensen (1999) Cytotechnology 30(1-3):71-83; Jordan et al. (1998) Cytotechnology 26(1):39-47; Lindell et al. (2004) Biochim. Biophys. Acta 1676(2):155-61). These large-scale cultures are generally grown in bioreactors, shakers, or incubators with stir plates, and may also be known as “spinner” or “suspension” cultures. Thus, as opposed to traditional transfections, in which cells are attached to plates or flasks, the disclosed method generally uses suspension cultures. Suspension cell cultures may be supported by regular media, such as Hams F12 or DMEM, or specialty media available from various commercial sources, such as FREESTYLE™ media available from Gibco (Gaithersburg, Md.), 293 SFM II™ media from Invitrogen (Carlsbad, Calif.), or SWIMCELL™ media available from bSYS (Basel, Switzerland). The choice of media for maintenance, transfection and expression will depend on the type of host cell used.

Transfecting cells requires optimization of several variables, including cell-seeding density (e.g., about 1×10⁵ to 3×10⁶ cells/ml culture), serum concentration (e.g., about 0% to about 10%, e.g., about 0.0001% to about 10%), incubation temperature (e.g., about 20-38° C.), transfection vehicle or reagent (chemical or electric), culture volume (e.g., about 50 ml to about 20 liters or more), and incubation time (e.g., about 24-144 hours). For each cell type, optimal parameters will vary. However, commercial suppliers generally provide optimization guidelines for particular cell types, as do various manuscripts that utilize transfection of the host cell chosen. These sources may be used to direct transfection of the chosen host cell, or may be used as a starting point from which simple trial and error may be used to provide optimum transfection parameters.

Labeling Proteins

The present invention discloses a method of producing a labeled protein by expressing the protein from the encoding polynucleotide in a host cell in the presence of labeled amino acids. Following transfection of a polynucleotide encoding the protein to be labeled, the host cells are cultured under conditions appropriate for protein expression. Such conditions are variable depending on a number of parameters, including but not limited to: the host cell used, the protein to be expressed, the type of labeling to be achieved, the size of the cell culture, and the expression construct used to drive expression of the protein. Optimum parameters for protein expression in host cells are extensively described in the literature or may be determined by simple trial and error (see, e.g., Gengross (2004) Nat. Biotechnol. 22(11): 1409-14; Culture of Animal Cells: A Manual of Basic Technique (4th ed.) ed. Freshney, Wiley-Liss, Alameda, Calif. (2000); General Techniques of Cell Culture in Handbooks in Practical Animal Cell Biology, eds. Harrison et al. Cambridge University Press, Boston, Mass. (1997)).

During the expression period, protein labeling is achieved by culturing recombinant host cells in the presence of labeled amino acids. As described above, various amino acids, e.g., natural, nonnatural, and analogs of amino acids, and various labels, e.g., heavy atom labels, isotope labels, fluorescent, chemiluminescent, or photolabile labels, or spin labels, may be used to label proteins of interest in the disclosed methods. The target amino acid position(s) chosen for substitution will vary depending on several parameters, for example: (1) whether the target amino acid positions are predicted to be, or are, involved in ligand binding, catalytic activity, and/or forming secondary or tertiary structure; (2) whether the residues of interest are predicted to be, or are, buried within the protein interior, a membrane, or a ligand-binding pocket; and (3) whether the labeled amino acid(s) occupying target amino acid position(s) are predicted to be, or are, proteolytically removed during protein processing. Additional considerations include the number of target positions available, the number of target positions to be labeled, and the technique or application requiring the labeled protein. It is within the knowledge of those skilled in the art to appropriately choose the target amino acid position(s) for appropriate labeling.

Protein labeling is achieved during expression of the protein of interest by culturing recombinant host cells in a chemically defined media. The disclosed methods may be used to label all, substantially all, or some target amino acid positions in the protein of interest. Therefore, the disclosed methods may be used to label less than all target amino acid positions within a protein of interest, i.e., it is possible to use the disclosed methods to partially label a protein of interest. Thus, if it is desirable to label substantially all or some positions occupied by a particular amino acid residue within a protein, the labeling medium may contain both labeled and unlabeled forms of that amino acid residue; any combination of ratios of labeled and nonlabeled amino acids is contemplated in the present invention. For example, one may label about 10% of the lysine positions within a protein by contacting the host cell with a labeling medium comprised of about 10% labeled lysine and 90% nonlabeled lysine. In some embodiments, the invention contemplates the use of labeling medium that is substantially free of the nonlabeled form of the labeled amino acid, as well as the use of labeling medium that contains a predetermined ratio of labeled to unlabeled amino acids.

In addition to labeling all, substantially all, or some of a single set of target amino acid positions within a desired protein, e.g., all, substantially all, or some methionine positions, it is possible to use the disclosed method to label multiple sets of target amino acid positions within a single protein, e.g., methionine and lysine positions. For example, if it is desirable to label both methionine and lysine positions within a protein of interest, then the labeling medium is supplemented with both labeled methionine and labeled lysine. In such an instance, the labeling medium may be substantially free of the unlabeled amino acids, e.g., unlabeled methionine and unlabeled lysine, or the labeling medium may contain predefined ratios of unlabeled to labeled amino acids, e.g., predefined ratios of unlabeled methionine to labeled methionine or unlabeled lysine to labeled lysine. Any combination of the above-identified examples is also contemplated as part of the invention, i.e., one may use the disclosed methods to simultaneously label substantially all positions occupied by, e.g., one type of amino acid, yet only some positions occupied by another type of amino acid(s). When using the disclosed methods to label more than one set of target amino acid position(s), e.g., methionines and lysines, the labels present on the labeled amino acid may be similar, e.g., heavy atom labels, the same, e.g., selenium labels, or different, e.g., a heavy atom and an isotope label.

Accordingly, the choice of labeling medium, and the contents of the labeling medium will depend on: (1) the target amino acid positions chosen for labeling; (2) the decision whether to label more than one set of target amino acid positions; and (3) the desired percent incorporation of labeled amino acid, i.e., at all, substantially all, or only some target amino acid positions. In one embodiment of the invention, the labeling medium is free or substantially free of the nonlabeled form of the labeled amino acid of choice. This allows the researcher to strictly regulate the level of unlabeled amino acid. Thus, if one wishes to label all or substantially all target amino acid position(s), one would add only labeled amino acid to the medium. Alternatively, if one wishes to label only some of the target amino acid positions, one would add a defined ratio of labeled to unlabeled amino acid to the medium. The culture medium, e.g., Hams F12, DMEM, MEM, RPMI, IPL-41, SF-900, FREESTYLE™, will depend on the type of host cell chosen, e.g., mammalian, fungi, yeast, or insect cells, and whether or not the host cell is derived from a primary source or a cell line. Additional ingredients in the labeling medium may include molecules designed to induce expression of the protein to be labeled. For example, the expression vector used to express the protein of interest for labeling may contain an inducible promoter such as the promoters present in, e.g., pCMV5-CymR or pCMV5(CuO) (Krakeler Scientific, Albany, N.Y.), pcDNA4/TO©, pcDNA4/TO/myc-His©, or pcDNA5/TO© (Invitrogen, Carlsbad, Calif.), and thus protein expression would require addition of, e.g., steroids, metals, alcohols or tetracycline. Additional ingredients typically found in culture media include buffers, antibiotics, antifungals, serum, etc., and will depend on the type of host cell used, the proposed culture time, the incubation temperature, and other variables that will be known to one skilled in the art. Various cell culture media that are free of specific amino acids are available from numerous commercial suppliers, e.g., methionine-free insect cell medium (Orbigen, San Diego, Calif.), and cysteine- and methionine-free mammalian medium (Sigma-Aldrich, St. Louis, Mo.).

In one embodiment, the medium is supplemented with serum, e.g., fetal bovine serum, fetal chick serum, fetal horse serum, etc. The type of serum chosen will depend on the type of host cell used, the proposed culture time, the incubation temperature, and other variables that will be known to one skilled in the art. Serum, which is enriched in growth factors used by growing and dividing cells, is typically used at concentrations from 1-15%. If desired, amino acid-free serum may be produced according to, e.g., the method of Dauphinais and Waithe (1977) J. Cell. Phys. 91:357-67. As disclosed herein, supplementing the labeling medium with higher concentrations of serum, i.e., about 10% serum, provides higher yields of the labeled protein of interest. Thus, a higher concentration of serum is often preferred.

Following suspension of the recombinant host cell in the appropriate labeling medium, the cells are incubated to allow incorporation of the labeled amino acid(s) into the target amino acid position(s) in the protein of interest. The time and temperature used for incubation will vary depending on the type of host cell chosen, the expression vector used to express the protein of interest, the protein to be expressed, the medium and serum used, and the labeled amino acid(s) to be incorporated into the protein of interest. For example, some labeled amino acids will be readily taken in and used by the host cell translation machinery, while other amino acids undergo slower uptake and lower incorporation into proteins. Additionally, some labeled amino acids may be cytotoxic, while others may induce degradation of the labeled protein. The promoter used in the expression vector, i.e., a high expression-inducing promoter, such as the CMV promoter, versus a low expression-inducing promoter, will also dictate protein expression levels. Further, the level of serum dramatically regulates protein expression in growing cells. In general, it is preferred to optimize the time of incubation to balance efficient incorporation of the labeled amino acid and high protein expression against labeled protein degradation and cytotoxicity. Culture temperatures will also vary depending on the host cell type, e.g., animal cells are generally maintained from about 31° C. to about 37° C., insect cells from about 20° C. to about 30° C., fungus from about 29° C. to about 33° C., and yeast from about 20° C. to about 26° C., although other culture temperatures can be used in the methods of the invention. In one embodiment of the invention, the host cells are incubated with labeling medium for about 48-144 hours at about 31° C.

Harvesting Labeled Proteins

The labeled protein is prepared by growing recombinant host cells under culture conditions that facilitate expression and labeling of the desired protein. The resulting labeled protein may then be purified from the culture medium or cell extracts for use in various biochemical techniques. Soluble forms of the labeled protein can be purified from conditioned media. Membrane-bound forms of labeled protein can be purified by preparing a total membrane fraction from the expressing cell and extracting the membranes with a nonionic detergent such as TRITON® X-100 (EMD Biosciences, San Diego, Calif.). Cytosolic or nuclear proteins may be prepared by lysing the host cells (via mechanical force, Parr-bomb, sonication, detergent, etc.), removing the cell membrane fraction by centrifugation, and retaining the supernatant.

The labeled protein can be purified using other methods known to those skilled in the art. For example, a labeled protein produced by the disclosed methods can be concentrated using a commercially available protein concentration filter, for example, an AMICON® or PELLICON® ultrafiltration unit (Millipore, Billerica, Mass.). Following the concentration step, the concentrate can be applied to a purification matrix such as a gel filtration medium. Alternatively, an anion exchange resin (e.g., a MonoQ column, Amersham Biosciences, Piscataway, N.J.) may be employed; such resin contains a matrix or substrate having pendant diethylaminoethyl (DEAE) or polyethylenimine (PEI) groups. The matrices used for purification can be acrylamide, agarose, dextran, cellulose or other types commonly employed in protein purification. Alternatively, a cation exchange step may be used for purification of labeled proteins. Suitable cation exchangers include various insoluble matrices comprising sulfopropyl or carboxymethyl groups (e.g., S-SEPHAROSE® columns, Sigma-Aldrich, St. Louis, Mo.).

The purification of the labeled protein from culture supernatant may also include one or more column steps over affinity resins, such as concanavalin A-agarose, AF-HEPARIN650, heparin-TOYOPEARL® or Cibacron blue 3GA SEPHAROSE® (Tosoh Biosciences, South San Francisco, Calif.); hydrophobic interaction chromatography columns using such resins as phenyl ether, butyl ether, or propyl ether; or immunoaffinity columns using antibodies to the labeled protein. Finally, one or more high performance liquid chromatography (HPLC) steps employing hydrophobic HPLC media, e.g., silica gel having pendant methyl or other aliphatic groups (e.g., Ni-NTA columns), can be employed to further purify the labeled protein. Alternatively, the labeled proteins may be recombinantly expressed in a form that facilitates purification. For example, the proteins may be expressed as a fusion with proteins such as maltose-binding protein (MBP), glutathione-S-transferase (GST), or thioredoxin (TRX). Kits for expression and purification of fusion proteins are commercially available from New England BioLabs (Beverly, Mass.), Pharmacia (Piscataway, N.J.), and Invitrogen (Carlsbad, Calif.), respectively. The labeled proteins can also be tagged with a small epitope (e.g., His, myc or Flag tags) and subsequently identified or purified using a specific antibody to the chosen epitope. Antibodies to common epitopes are available from numerous commercial sources. Some or all of the foregoing purification steps, in various combinations or with other known methods, can be employed to purify a labeled protein.

Use of Labeled Proteins

Labeled proteins produced according to the disclosed methods may be used in various biochemical and molecular biology techniques such as, e.g., spectroscopy, microscopy, and crystallography, and applications including protein structure determinations, protein tracing and/or localization, diagnostic and therapeutic applications, and affinity experiments. Such applications and techniques utilize physical chemistry, biochemistry and molecular biology methods that include, but are not limited to, X-ray diffraction crystallography (see, e.g., Liu and Hsu (2005) Proteomics 5(8):2056-68); X-ray absorption spectroscopy (see, e.g., Chen (2005) Ann. Rev. Phys. Chem. 56:221-54); multiwavelength anomalous dispersion (MAD) (see, e.g., Hendrickson et al., supra); single-wavelength anomalous dispersion (SAD) (see, e.g., Dauter (2002) Curr. Opin. Struct. Biol. 12(5):674-78); multiple isomorphous replacement (MIR) (see, e.g., Dauter, supra); electron paramagnetic resonance (EPR) (see, e.g., Mobius (2000) Chem. Soc. Rev. 29:129-39); nuclear magnetic resonance (NMR) (see, e.g., Weigelt et al. (2002) J. Am. Chem. Soc. 124(11):2446-47); mass spectrometry (MS) (see, e.g., Gruhler et al., supra; Martinovic et al. (2002) J. Mass Spectrom. 37(1):99-107); circular dichroism (CD) (see, e.g., Pelton and McClean (2000) Analyt. Biochem. 277:167-72); electron spin resonance (ESR) (see, e.g., Hoffman (2003) Proc. Natl. Acad. Sci. USA 100(7):3575-78); infrared spectroscopy (IR) (see, e.g., Pelton and McClean, supra); surface plasmon resonance (SPR) (see, e.g., Buijs and Franklin (2005) Brief Funct. Genomic Proteonomic 4(1):39-47); electron nuclear double resonance (ENDOR) (see, e.g., Hoffman, supra); electron-electron double-resonance (ELDOR) (see, e.g., Fajer, supra; Bennati et al. (2003) J. Am. Chem. Soc. 125 (49): 14988-89); electron-spin-echo-envelope-modulation (ESEEM) (see, e.g., Hoffman, supra); Raman spectroscopy (RS) (see, e.g., Eikje et al. (2005) J. Biomed. Opt. 10(1):14013); electron microscopy (EM) (see, e.g., Neelissen et al. (1999) Microscopy Res. Tech. 47(4):286-90); fluorescence correlation spectroscopy (FCS) (see, e.g., Kohl and Schwille (2005) Adv. Biochem. Eng. Biotechnol. 95:107-42); confocal microscopy (CF) (see, e.g., Tsien and Waggoner, in Handbook of Confocal Microscopy (2nd ed.) ed. Pawley, Plenum Press, NY (1994)); fluorescence resonance energy transfer microscopy (FRET) (see, e.g., Molecular Imaging: FRET Microscopy and Spectroscopy, eds. Periasamy and Day, Oxford University Press (2005)); hyperfine sublevel correlation spectroscopy (HYSCORE) (see, e.g., Martinez et al. (1997) Biochemistry 36:15526-37); fluorescence lifetime image microscopy (FLIM) (see, e.g. Wallrabe and Periasmay (2005) Curr. Opin. Biotechnol. 16(1): 19-27); fluorescent speckle microscopy (FSM) (see, e.g., Waterman-Storer and Danuser (2002) Current Biology 12:R633-40); total internal reflection fluorescence microscopy (TIRF) (see, e.g., Axelrod et al. (1983) J. Microsc. 129(1):19-28); positron emission tomography (PET) (see, e.g., Weiner and Thakur, supra); SIDEC™ electron tomography (SET) (see, e.g., Banyay et al. (2004) Assay Drug Develop. Tech. 2(5):561-67); and atomic force microscopy (AFM) (see, e.g., Scheuring et al. (2005) Biochim. Biophys. Acta. 1712(2): 109-27).

Structural Determinations Using Labeled Proteins

Labeled proteins produced by the disclosed methods are useful in structural determinations. As used herein, the phrase “structural determinations” refers to identifying or aiding in the identification of the three-dimensional structure of a labeled protein or a domain (or motif) of a labeled protein. Structural determination techniques are well known in the art and include, e.g., crystallography, microscopy and spectroscopy techniques.

Depending on the labeled amino acid incorporated into the protein of interest, various procedures may be used to carry out structural determinations. For example, isotopically labeled amino acids are often used for NMR studies, while heavy atom labeled amino acids are commonly employed by MAD and crystallography techniques. In addition, spin-labeled amino acids are used for methods that require unpaired electrons to align with a magnetic field (such as ESEEM, ENDOR and EPR). However, it will be understood by one skilled in the art that these categories are not fixed, e.g., paramagnetic spectroscopy methods such as ESEEM, ENDOR, and EPR, can use spin-labeled amino acids as well as isotope-labeled amino acids.

Crystallography, microscopy, and spectroscopy studies that may employ a labeled protein produced by the disclosed methods include, but are not limited to, X-ray diffraction, X-ray absorption, multiwavelength anomalous dispersion (MAD), single-wavelength anomalous dispersion (SAD), multiple isomorphous replacement (MIR), electron paramagnetic resonance (EPR), nuclear magnetic resonance (NMR), mass spectrometry (MS), circular dichroism (CD), electron spin resonance (ESR), surface plasmon resonance (SPR), electron nuclear double resonance (ENDOR), electron-electron double-resonance (ELDOR), electron spin-echo-envelope-modulation (ESEEM), Raman spectroscopy (RS), electron microscopy (EM), fluorescence correlation spectroscopy (FCS), confocal microscopy (CF), immunofluorescence microscopy (IF), fluorescence resonance energy transfer microscopy (FRET), hyperfine sublevel correlation spectroscopy (HYSCORE), fluorescence lifetime image microscopy (FLIM), fluorescent speckle microscopy (FSM), total internal reflection fluorescence microscopy (TIRF), positron emission tomography (PET), SIDECTM electron tomography (SET), and atomic force microscopy (AFM).

Protein Localization and Tracing, Diagnostic and Therapeutic Applications, and Affinity Experiments Using Labeled Proteins

Labeled proteins produced by the disclosed methods are useful in protein tracing studies and protein localization studies. As used herein, the phrase “protein tracing” refers to: (1) following the migration of a labeled protein within or between a cell(s), tissue(s), or within an organism; and/or (2) following and/or analyzing the metabolic breakdown, turnover, or degradation of a protein within a cell, tissue, or organism. As used herein, the phrase “protein localization” or “protein localizing” refers to identifying or imaging the position, expression level (as discussed in Gruhler et al. supra, i.e., quantitative proteomic analysis of selectively labeled proteins), or accumulation of a particular protein, e.g., within a cellular compartment or organelle, tissue or organism.

For example, a labeled form of a protein may be used to trace the metabolism of a protein of interest, e.g., the conversion of a protein precursor (such as a propeptide or prepropeptide) to a product (such as a peptide), or the breakdown/turnover of the protein within a cell, tissue, or organ (see, e.g., Camier et al. (1986) FEBS Lett. 196(1):14-18; Stahelin, supra; Mitch and Clark, supra). A labeled form of a protein may be used to trace the migration of a protein within or between cell(s) and tissue(s), or within an organism (see, e.g., Dadoune et al. (1985) Arch. Androl. 14(2-3):199-207; Chaurand et al. (2005) Toxicol. Path. 33:92-101; Hollenbeck (1989) J. Cell Biol. 108:223-27; Kues et al. (2001) Biophys. J. 80:2954-67; Patel et al., supra; Easty et al., supra; Holstege and Kuypers, supra; Kirkpatrick et al., supra). Additionally, labeled proteins may be used to locate the position of a protein within cells, tissues, and organs (see, e.g., Kenworthy (2001) Methods 24:289-96; Wouters et al. (1998) EMBO J. 17:7179-89; Chamberlain and Hahn (2000) Traffic 1:755-62).

Isotope-labeled proteins produced by the disclosed methods may be used for diagnostic and therapeutic applications. As used herein, the phrase “diagnostic and therapeutic applications” refers to using a labeled protein produced by the disclosed methods to diagnose, prognose, monitor, treat, ameliorate or prevent a disorder treatable by radioisotopes, e.g., systemic or localized cancers. For example, a radiolabeled protein produced by the invention may be used to direct radioisotopes to cancer lesions, cancerous cells, or a desired tissue or organ for treatment or detection of cancerous or precancerous pathologies (see, e.g., Weiner and Thakur, supra).

Labeled proteins produced by the disclosed methods are also useful in affinity studies. As used herein, the phrase “affinity studies” refers to examining the interaction of a labeled protein with molecules or cells, such as, e.g., antibodies, polynucleotides, antigens, receptors, ligands, etc., or the formation of protein complexes. For example, a labeled form of a protein may be used to analyze the interaction of an antibody with an antigen, a receptor with a ligand, cell-cell interactions via receptors or matrix molecules, cytoskeletal protein interactions, transcription factor interactions with polynucleotides, protein dimerization, or protein interactions with therapeutic moieties (see, e.g., Yan and Marriott (2003) Curr. Opinion Chem. Biol. 7:635-40; Kenworthy, supra; Hollenbeck, supra).

Depending on the labeled amino acid incorporated into the protein of interest, various techniques may be used to carry out protein localization, protein tracing, or affinity studies. For example, FRET, FLIM, FSM, FCS, TIRF, EM, PET, SET, and CF may be used to detect fluorescently labeled proteins, heavy atom-labeled proteins, or isotope-labeled proteins within cell compartments and organelles or within tissues, or to map protein organization in plasma membranes (see, e.g., Waterman-Storer and Danuser (2002) Current Biology 12:R633-40; Tsien and Waggoner, supra; Kenworthy, supra; Bastiaens and Jovin (1996) Cell Biol. 93:8407-12; Melan and Sluder (1992) J. Cell Sci. 101:731-43; Axelrod et al. (1984) Ann. Rev. of Biophys. Bioeng. 13:247-68; Neelissen et al., supra). Microscopy studies employing photolabile or fluorescently labeled proteins may also be used to detect protein interactions within adherens junctions, centrosomes, peroxisomes and cell-cell contact zones, as well as receptor-ligand interactions (see, e.g., Periasamy and Day, supra; Kenworthy, supra; Wouters et al. (1998) EMBO J. 17:7179-89).

In addition to microscopy methods, labeled proteins may be used for protein tracing, protein localization, and affinity studies by obtaining samples (e.g., cells, cell extracts, cell membranes, cell fractions, or tissues previously supplied with labeled proteins produced by the disclosed methods) and subjecting the samples to scintillation counting, flow cytometry, PET, SET, histology, autoradiography, and other well-known techniques that allow imaging and/or identification of a labeled protein within a sample (see, e.g., von Banchet and Heppelmann (1995) J. Histo. Cyto. 43:821-27; Banyay et al. (2004) Assay Drug Develop. Tech. 2(5):561-67; Weiner and Thakur, supra). Additionally, for affinity studies, labeled proteins may be identified within a sample and then isolated and subjected to immunoprecipitation, SDS-PAGE, and immunoblotting to identify binding partners. Such methods may be found in numerous microbiology, cellular biology and biochemistry texts, e.g., Sambrook et al., supra. Protein tracing, protein localization, and affinity studies using the labeled proteins produced by the disclosed methods, may be performed in cell-free systems, in vitro (e.g., in cell culture), ex vivo (e.g., in cell explants), or in vivo (e.g., in an intact organism).

The entire contents of all references, patents, patent applications, and other patent documents cited throughout this application are hereby incorporated by reference herein.

### EXAMPLES

The Examples which follow are set forth to aid in the understanding of the invention but are not intended to, and should not be construed to, limit the scope of the invention in any way. The Examples do not include detailed descriptions of conventional methods, such as recombinant DNA techniques. Such methods are well known to those of ordinary skill in the art.

### Example 1

### Creation of Transiently Transfected HEK293 Cells Maintained as Large-Scale Spinner Cultures

### Example 1.1

### Cell Lines and Cell Culture

Mammalian HEK293-EBNA cells were grown and maintained in a humidified incubator with 5% CO₂ at 37° C. HEK293 cells were cultured in FREESTYLE™ 293 media (Invitrogen, Carlsbad, Calif.) (hereinafter “293 media”) supplemented with 5% fetal bovine serum (FBS).

### Example 1.2

### Large-scale Transient Expression for Selenomethionine Labeling

Transient expression was performed in either 50 ml spinners or 1 L spinners. For 50 ml cultures, 25 μg of plasmid DNA (described below) was mixed with 400 μg of polyethylenimine [(PEI) 25 kDa, linear, neutralized to pH 7.0 by HCl (1 mg/ml), Polysciences (Warrington, Pa.)] in 2.5 ml of serum-free 293 media. For 1 L cultures, 500 μg of DNA was mixed with 4 mg of PEI in 50 ml of serum-free 293 media. The plasmid/PEI/medium aliquots were then combined in spinner flasks with either 50 ml or 1 L of HEK293 cells (1.5×10⁶ cells/ml final culture volume) in 293 media supplemented with 5% FBS. After 24-48 hours, the transfection media was removed and the cells were washed once with PBS buffer prior to labeling. Cells were resuspended in the labeling media [methionine-free and cysteine-free DMEM (Invitrogen), 50 mg/L selenomethionine, 300 mg/L cysteine, 2 mM glutamine, 5% or 10% FBS] and incubated at 37° C. with a rotation rate of 170 rpm on a P2005 Stirrer (Bellco, Amityville, N.Y.) for 48-120 hours.

### Example 1.3

### Vectors and DNA Constructs

The backbone vector used for cloning of the IKK2 polynucleotide was the pSMEDA vector (an in-house mammalian expression vector, see U.S. Provisional Application No. 60/672,997, incorporated herein by reference in its entirety). PCR, using primer WZ203 (5′-gctctagattacttgctacaagcaatcttcaggag-3′) (SEQ ID NO:1) and primer WZ138 (5′-gctctagagccgccaccatgcatcatcatcatcatcatagctggtca-3′) (SEQ ID NO:2), was performed on a template pWZ1033 (encompassing a cDNA of human IKK2). The PCR product, which encodes the 664 amino acid residues of IKK2 and a C-terminal His6 tag (see FIG. 1 (SEQ ID NO:3)), was digested with Xba1 and EcoR1, and subcloned into the pSMEDA vector. To produce the IKK2 expression construct, an N-terminal His6 tag was incorporated by PCR into the pSMEDA vector downstream of the methionine start codon.

### Example 2

### Labeling of IKK2 Transiently Expressed in Large-Scale HEK293 Spinner Cultures

### Example 2.1

### Incubation of Recombinant Host Cells Expressing IKK2 with Labeling Medium

Spinner cultures of transient transfectants were incubated at 37° C. with a rotation rate of 170 rpm on a P2005 Stirrer for 24 hours. The cells were pelleted and resuspended in labeling medium (methionine-free and cysteine-free DMEM (Invitrogen, Carlsbad, Calif.), 50 mg/L selenomethionine, 300 mg/L cysteine, 2 mM glutamine, 5% or 10% FBS). The cultures were grown at 31° C. for 48-120 hours before harvesting labeled IKK2 protein.

### Example 2.2

### Purification of IKK2

IKK2-expressing HEK293 cells pelleted from 1 liter of labeling culture were resuspended in lysis buffer (50 mM Hepes pH 7.5, 100 mM NaCl, 5 mM β-mercaptoethanol, 5% glycerol, 15 mM imidazole, 5 mM benzamidine, 5 mM β-glycerol phosphate disodium, protease inhibitors [EDTA-free cocktail tablets (Roche Diagnostic, Nutley, N.J.), DNase, and RNase]. Cells were lysed using a Parr-Bomb apparatus (Parr Instrument Company, Moline, Ill.) and the lysates were centrifuged at 12K rpm (Sorvall rotor, Kendro Laboratory Products, Newton, Conn.) for 15 min at 4° C. Cell supernatants were loaded onto a Ni-NTA column attached to an HPLC analyzer. After a 30-column-volume wash with lysis buffer containing 15 mM imidazole, protein samples were eluted with 200 mM imidazole. The elution was diluted four-fold using 25 mM Hepes pH 7.5, 10 mM NaCl, 5 mM DTT, 5% glycerol and 5 mM β-glycerol phosphate disodium, and loaded onto an AF-Heparin-650M affinity column (Tosoh Biosciences, South San Francisco, Calif.). Protein samples were eluted from the column using a 0-1M NaCl gradient in 25-column volumes. Heparin fractions containing IKK2 were diluted ten-fold using 25 mM Hepes pH 7.5, 10 mM NaCl, 5% glycerol, 5 mM DTT, and 5 mM P-glycerol phosphate disodium. Protein samples were further purified with a strong anion exchange column (MonoQ, Amersham Bioscience, Piscataway, N.J.). Protein samples were eluted from the column using a 0-1M NaCl gradient in 25-column volumes. Protein concentration was determined by spectroscopy at 280 nm, and the purity was observed by SDS-PAGE. SDS-PAGE gels were subjected to immunoblotting with mouse monoclonal anti-His4 antibody (FIG. 2). Samples from fractions eluted from the MonoQ column were also separated by SDS-PAGE and stained with Coomassie Blue to determine purity (FIG. 3).

As shown in FIG. 2, recombinant IKK2 protein is located in both pellet (P) and supernatant (S) fractions from transiently transfected cells. Although a larger quantity of IKK2 is produced by HEK293 cells grown in either 293 medium or S-MET medium, soluble IKK2 is clearly produced in HEK293 cells grown in Se-MET-labeling medium. As shown in FIG. 3, the final protein yield of IKK2 is about 30 μg per liter of culture as estimated by absorbance at 280 nm and comparison to known loading controls (left lane).

To optimize protein yields, transfected HEK293 cells were resuspended in Se-MET-labeling medium with 10% FBS, and incubated at 31° C. for an additional 48-120 hours. As shown in FIG. 4, expression of soluble Se-MET-labeled IKK2 is significantly higher in the presence of 10% FBS than with 5% FBS (e.g., compare FIG. 4, lanes 3, 4 with FIG. 2, lane 5; compare FIG. 4, lanes 7, 8 with FIG. 2, lane 6). Thus, the expression of soluble Se-MET-labeled IKK2 can be improved by higher serum concentration.

### Example 3

### Spectrophotometry Analysis of Se-MET-labeled IKK2

The incorporation of selenomethionine into IKK2 recombinantly produced in HEK293 cells (described in Examples 1 and 2) was analyzed by HPLC ESI-Mass Spectrum. C4 HPLC ESI-MS was used to monitor and compare the biotransformation of the normal methionine (S-MET) containing N-acetyl IKK protein to the selenium methionine (Se-MET) form.

The Se-MET-IKK protein (K664) has a MW of 77,396 Da and is phosphorylated at five sites. The mass difference between Se (79 Da) and S (32 Da) is 47 Da. The Se-MET N-acetyl IKK was produced with a MW of 78,330 Da, corresponding to a mass shift of 934 Da, consistent with the substitution of 20 residues of Se-MET for S-MET, the predicted number of methionines in the IKK protein. As shown in FIG. 5, selenomethionine-labeled IKK2 is produced in large-scale transiently transfected HEK293 cultures (with incorporation of selenomethionine at about 80% of available methionine positions).

What is claimed is:

1. A method of selectively labeling a protein at a target amino acid position(s) comprising the steps of:
(a) transiently transfecting a host cell in a large-scale cell culture with a polynucleotide encoding a protein of interest;
(b) contacting the cell with a labeling medium, wherein the labeling medium contains a labeled amino acid(s) to be incorporated into the protein at the target amino acid position(s); and
(c) expressing the protein of interest under conditions suitable to allow incorporation of the labeled amino acid into the target amino acid position(s),
thereby labeling the protein at a target amino acid positions(s).

2. The method according to claim 1, wherein the labeled amino acid in the labeling medium is derived from either a naturally occurring or nonnaturally occurring amino acid.

3. The method according to claim 1, wherein the labeled amino acid in the labeling medium contains a label selected from the group consisting of a heavy atom label, a fluorescent label, a chemiluminescent label, a photolabile label, an isotope label, and a spin label.

4. The method according to claim 3, wherein the labeled amino acid in the labeling medium contains a heavy atom label, and wherein the heavy atom label is selenium.

5. The method according to claim 1, wherein the labeled amino acid in the labeling medium is selenomethionine.

6. The method according to claim 1, wherein the host cell is selected from the group consisting of a mammalian cell, a fungal cell, an insect cell, and a yeast cell.

7. The method according to claim 6, wherein the host cell is a mammalian cell, and wherein the mammalian cell is selected from the group consisting of HEK293 cells and CHO cells.

8. The method according to claim 1, wherein cell density during the transfecting step is about 1×10⁵ to about 3×10⁶cells/ml.

9. The method according to claim 1, wherein the volume of culture medium in the large-scale cell culture in the transfecting step is greater than about 50 ml.

10. The method according to claim 1, wherein the volume of culture medium in the large-scale cell culture in the transfecting step is greater than about 1 liter.

11. The method according to claim 1, wherein the transfecting step uses a technique selected from the group consisting of polyethylenimine transfer, electroporation, calcium phosphate precipitation, liposomal transfer, gene gun transfer, DEAE-dextran transfer, and episomal transfer.

12. The method according to claim 11, wherein the transfecting step uses the technique of polyethylenimine transfer.

13. The method according to claim 1, wherein the transfecting step proceeds for about 24-144 hours.

14. The method according to claim 1, wherein the contacting step is carried out in a labeling medium supplemented with serum, and wherein the labeling medium substantially lacks a nonlabeled form of the labeled amino acid.

15. The method according to claim 14, wherein the serum is present in the labeling medium at about 0.0001% to about 10%.

16. The method according to claim 1, wherein the expressing step proceeds at a temperature in the range of about 20° C. to about 38° C.

17. The method according to claim 1, wherein the expressing step proceeds for about 24-144 hours.

18. The method according to claim 1, wherein the labeled amino acid is incorporated at about 50-95% of the corresponding amino acid positions in the labeled protein.

19. A method of using a labeled protein produced according to the method of claim 1, wherein the method of using the labeled protein is selected from the group of techniques consisting of microscopy, spectroscopy, and crystallography.

20. A method of using a labeled protein produced according to the method of claim 1, wherein the method of using the labeled protein is selected from the group of techniques consisting of X-ray diffraction, X-ray absorption, multiwavelength anomalous dispersion (MAD), single-wavelength anomalous dispersion (SAD), multiple isomorphous replacement (MIR), electron paramagnetic resonance (EPR), nuclear magnetic resonance (NMR), mass spectrometry (MS), circular dichroism (CD), electron spin resonance (ESR), surface plasmon resonance (SPR), electron nuclear double resonance (ENDOR), electron-electron double-resonance (ELDOR), electron spin-echo-envelope-modulation (ESEEM), Raman spectroscopy (RS), electron microscopy (EM), fluorescence correlation spectroscopy (FCS), confocal microscopy (CF), immunofluorescence microscopy (IF), fluorescence resonance energy transfer microscopy (FRET), hyperfine sublevel correlation spectroscopy (HYSCORE), fluorescence lifetime image microscopy (FLIM), fluorescent speckle microscopy (FSM), total internal reflection fluorescence microscopy (TIRF), positron emission tomography (PET), Sidec electron tomography (SET), and atomic force microscopy (AFM).

21. A method of using a labeled protein produced according to the method of claim 1, wherein the method of using the labeled protein is selected from the group of applications consisting of protein structure determinations, protein tracing and/or localization, diagnostic and therapeutic applications, and affinity experiments.

22. A labeled protein produced according to the method of claim 1.


SEQUENCE LISTING (3 sequences)

SEQ ID NO: 1 - DNA, 35 bp, Artificial
  PCR primer WZ203
gctctagattacttgctacaagcaatcttcaggag

SEQ ID NO: 2 - DNA, 47 bp, Artificial
  PCR primer WZ183
gctctagagccgccaccatgcatcatcatcatcatcatagctggtca

SEQ ID NO: 3 - protein, 670 aa, Artificial
  PCR product [His6-tagged human IKK2 (deltaK664)]
MHHHHHHSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSK